(19) United States
(12) Patent Application Publication
(10) Pub. No.: US 2011/0104280 A1
(43) Pub. Date: May 5, 2011

(54) Wound treatment systems, devices, and methods using biocompatible synthetic hydrogel compositions

(76) Inventor: Olexander Hnojewyj, Redwood City, CA (US)

(21) Appl. No.: 12/800,680

(22) Filed: May 20, 2010

Related U.S. Application Data
(63) Continuation-in-part of application No. 12/454,593, filed on May 20, 2009.

(51) Int. Cl. A61K 9/00; A61P 17/02; A61B 17/03

(52) U.S. Cl. 424/486; 606/214

(57) ABSTRACT

A multi-arm poly(ethylene glycol) (PEG) Succinimidyl Glutarate is mixed with a biocompatible, synthetic, nucleophilic polymer component essentially free of human or bovine albumin and other biological molecules, containing, e.g., a polypeptide moiety having a number of active surface lysines of at least twenty (20) per 5000 M/W, which can also be blended with a multi-arm poly(ethylene glycol) (PEG) Amine. The mixture forms a synthetic hydrogel composition. The synthetic hydrogel composition can be applied by topically spraying the synthetic hydrogel composition onto a targeted wound site to promote wound healing.

### RELATED APPLICATIONS

This application is a continuation-in-part of co-pending U.S. patent application Ser. No. 12/454,593, filed May 20, 2009, entitled “Vascular Puncture Closure Systems, Devices, and Methods Using Biocompatible Hydrogel Compositions,” which is incorporated herein by reference. This application also claims the benefit of U.S. Provisional Patent Application Ser. No. 61/275,534, filed Aug. 31, 2009, entitled “Wound Treatment Systems, Devices, and Methods Using Biocompatible Synthetic Hydrogel Compositions,” and U.S. Provisional Patent Application Ser. No. 61/337,294, filed Feb. 20, 2010, entitled “Wound Treatment Systems, Devices, and Methods Using Biocompatible Synthetic Hydrogel Compositions,” both of which are incorporated herein by reference.

### FIELD THE INVENTION

The invention relates to biocompatible materials and additives that are formulated for biomedical applications, such as wound healing and hemostasis.

### BACKGROUND OF THE INVENTION

Hemostatic agents are used to stop or control bleeding by either promoting coagulation or contacting tissue. The bleeding may be caused by trauma, e.g. organ (liver, kidney) lacerations, or may be caused during surgery, e.g. cyst removal, bone bleeding, or burn operations.

Bleeding is usually controlled by the application of synthetic or natural sheets of gauze and Gelfoam™ material or Sugicel™ material. These materials, in certain procedures, are soaked with a hemostatic agent, such as thrombin or epinephrine, or formulations of sprayable fibrin adhesive.

In some situations, conventional hemostasis treatments achieve clinically acceptable time. Still, there are many drawbacks.

As an example, fibrin adhesives and Gelfoam™ are formulated with bovine thrombin and collagen, respectively, to cause the desired clotting response. These biologic materials all have the potential for the transmission of bovine spongiform encephalopathy—“Mad Cow Disease”—to humans. Further, problems such as intraoperative blood loss, lack of hemostasis, engraftment (failure of skin grafts), adherence, and less than satisfactory cosmetic results still persist.

Certain surgical procedures and traumatic injuries are prone to massive blood loss. In these circumstances, conventional approaches for dealing with blood loss, such as manual pressure, cauterization, or sutures can be time consuming and ineffective. More recent advances include products such as hemostatic agents and tissue sealants. Hemostats stop bleeding by initiating or accelerating the body's clotting process, mechanically or chemically. They are indicated to help contain bleeding during surgery and minimize postoperative re-bleeding and oozing. Though widely used and available in many forms, such products are ineffective in patients with profuse hemorrhage or compromised clotting mechanisms—most commonly due to the consumption of coagulation factors (coagulopathy), disease (haemophelia, von Willibrand), or medication (oral anticoagulants), the latter of which comprises a significant and rapidly growing demographic of the patient population.

Autologous and allogenic fibrin sealants are now available as an alternative to hemostatic agents and do not rely on the full complement of blood factors to produce hemostasis. Topical tissue sealants act as a physical barrier to blood loss by sealing wounds and potentially aiding in healing. The products are two-part liquid systems that react when mixed to create a physical barrier to blood loss. They are typically applied using two syringes coupled with a mixing chamber and delivery nozzle or tube. Fibrin sealants, which are the most commonly used, combine biological proteins, thrombin and fibrinogen. These require patient sensitivity testing, are difficult to prepare, and present a risk of transmitting infection. Furthermore, lot-to-lot performance is extremely inconsistent due to inherent biological variability.

For severe trauma (e.g. battlefield, accident, violence, sports) and surgical procedures characteristic of profuse bleeding (e.g. burn grafting, liver transplant), the critical shortcomings of sealants are rapid, uncontrollable reaction (set) timing which impedes distribution and promotes delivery system clogging, the notorious failure to provide adequate adhesion in wet environments, and the combined impact of both which leads to the tendency to be washed away upon delivery.

Despite conventional treatments for hemostatic barriers and surgical adhesives, there is a need for synthetic biomaterials that safely, quickly, and reliably stop or control fluid leakage in body tissues without necessarily activating the patient's coagulation pathways.

### SUMMARY OF THE INVENTION

The invention provides compositions, systems, instruments, and methods for creating families of synthetic biocompatible, hydrogel compositions that can be used in diverse therapeutic indications, among them being wound healing and the arrest or control of bleeding or leakage of fluid in body tissue. By “synthetic,” it is meant that the component is chemically synthesized in the laboratory or industrially or produced using recombinant DNA technology. The term “hydrogel” or “hydrogel composition” refers to a state of matter comprising a cross-linked polymer network swollen in a liquid medium. According to this aspect of the invention, the hydrogel transforms over time by physiologic mechanisms from a solid state back to a biocompatible liquid state, which can be cleared by the body. The transformation can occur, e.g., by hydrolysis of the polymer backbone.

One representative aspect of the invention comprises a biocompatible, synthetic, electrophilic (i.e., electron withdrawing) polymer component mixed with a biocompatible, synthetic, nucleophilic (i.e., electron donating) polymer component. When mixed, the components cross-link to form the synthetic biocompatible, hydrogel composition.

The electrophilic component and/or the nucleophilic component can include additive components, which can affect the physical and mechanical characteristics of the composition.

Another representative aspect of the invention provides a system for promoting wound healing. The system comprises a first solution, a second solution, and instructions for mixing the first and second solutions for use. The first solution comprises a biocompatible, synthetic, electrophilic polymer component including a multi-arm poly(ethylene glycol) (PEG) Succinimidyl Glutarate. The second solution comprises a biocompatible, synthetic, nucleophilic polymer component essentially free of human or bovine albumin and other biological molecules and including a polypeptide moiety having a number of active surface lysines of at least twenty (20) per 5000 M/W, and optionally blended with a multi-arm poly(ethylene glycol) (PEG) Amine. The instructions for use comprise mixing the first and second solutions to form a synthetic hydrogel composition, and applying the synthetic hydrogel composition by topically spraying the synthetic hydrogel composition onto a targeted wound site to promote wound healing.

In one embodiment the polypeptide moiety comprises Poly-L-Lysine hydrobromide.

In one embodiment, the multi-arm poly(ethylene glycol) (PEG) Succinimidyl Glutarate has a functionality of four.

In one embodiment, the optional multi-arm poly(ethylene glycol) (PEG) Amine (PEG-Amine) has a functionality of four.

In one embodiment, the system further includes one more auxiliary component comprising salicylate-based polyanhydride-esters formulated to degrade and release salicylic acid for anti-inflammatory effect; fillers, such as glucosamine, glucosaminoglycans, and chondroitin sulfate; anti-inflamatory drugs; rapamycines and analogs, such as everolimus and biolimus; dexamethasone; M-prednisolone; interferon γ-1b; leflunomide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG; plasticizers, including cellulose and/or non-reactive PEG compounds, such as PEG-hydroxyl compounds; therapeutic agents such as stem cells, antibodies, antimicrobials, collagens, genes, DNA, and other therapeutic agents; hemostatic agents such as thrombin, chitosan, diatomaceous earth (CELOX Material), silver, and/or GELFOAM® Material; growth factors; vasoconstrictors such as Ephinephrine (which includes hydroxyl groups (—OH) and an amine group (—NH) that could be incorporated to react with PEG-SG); lydocaine; and comparable compounds.

Another example of auxiliary components that can be added include an iodinated moiety such as providone-iodine (polyvinylpyrrolidone and iodine) for antifungal/antibacterial topical application and wound healing.

Another example of auxiliary components that can be added include aloe vera, also known as the medicinal alow, which contains iodine, for wound healing.

Another example of auxiliary components that can be added include fluorocarbons (fluorine substituted hydrocarbons) and perfluorocarbons (fluorocarbons in which all of the hydrogen atoms have been replaced with fluorine), such as perfluorodecalin (CAS No. 306-94-5) and perfluorthributylamine. These compounds, because of their ability to dissolve large amounts of oxygen, can be applied topically, to provide extra oxygen to a specific location, to accelerate wound healing.

With current state of the art emulsion technology, fluoro-materials can be incorporated into the hydrogel composition. Fluoro-materials can be used to pressurize delivery units for the first and/or second components, to mix and deliver the hydrogel composition with O2 (oxygen) to the wound site.

In one embodiment, the system further includes a dispensing unit that mixes the first and second solution and dispenses the mixture in situ through a dispensing tip. In this arrangement, the instructions for use direct use of the dispensing unit.

In one embodiment, the dispensing unit is sized and configured as an integrated hand held device, or as an integrated hand held endoscopic device, or as an instrument system having mixing and dispensing units.

In one embodiment, the dispensing tip is sized and configured as a needle, sprayer, or atomizer.

Another representative aspect of the invention provides a method for promoting wound healing. The method comprises (i) providing a first solution comprising a biocompatible, synthetic, electrophilic polymer component including a multi-arm poly(ethylene glycol) (PEG) Succinimidyl Glutarate; (ii) providing a second solution comprising a biocompatible, synthetic, nucleophilic polymer component essentially free of human or bovine albumin and other biological molecules and including a polypeptide moiety having a number of active surface lysines of at least twenty (20) per 5000 M/W, and optionally blended with a multi-arm poly(ethylene glycol) (PEG) Amine (PEG-Amine); (iii) mixing the first and second solutions to form a synthetic hydrogel composition; and (iv) applying the synthetic hydrogel composition by topically spraying the synthetic hydrogel composition onto the wound site to promote wound healing.

Another representative aspect of the invention provides a system for wound healing. The system comprise a first solution, a second solution, and instructions for mixing the first and second solutions for use. The first solution comprises a biocompatible, synthetic, electrophilic polymer component including a poly(ethylene glycol) (PEG) Succinimidyl Glutarate having a functionality of four and a molecular weight of about 10,000 g/mole. The second solution comprises a biocompatible, synthetic, nucleophilic polymer component essentially free of human or bovine albumin and other biological molecules and including Poly-L-Lysine hydrobromide having a number of active surface lysines of at least twenty (20) per 5000 M/W blended with a poly(ethylene glycol) (PEG) Amine having a functionality of four and a molecular weight of about 10,000 g/mole, wherein the weight-to-weight ratio of poly(ethylene glycol) (PEG) Amine to poly(ethylene glycol) (PEG) Succinimidyl Glutarate is selected to be about 0.7 to 1.0. The instructions for use comprise mixing the first and second solutions to form a synthetic hydrogel composition, and applying the synthetic hydrogel composition by topically spraying the synthetic hydrogel composition onto a targeted wound site to promote wound healing.

In one embodiment, the first solution is essential free of a buffer material.

In one embodiment, the first solution comprises poly(ethylene glycol) (PEG) Succinimidyl Glutarate dissolved in Sterile Water for Injection USP (SWI) essentially free of a buffer material.

In one embodiment, the second solution comprises the Poly-L-Lysine hydrobromide and poly(ethylene glycol) (PEG) Amine dissolved in HPLC-grade water for delivery that includes a buffer material.

In one embodiment, the system further includes a dispensing unit that mixes the first and second solution and dispenses the mixture in situ through a dispensing tip. In this arrangement, the instructions for use direct use of the dispensing unit.

In one embodiment, the dispensing unit is sized and configured as an integrated hand held device, or as an integrated hand held endoscopic device, or as an instrument system having mixing and dispensing units.

In one embodiment, the dispensing tip is sized and configured as a needle, sprayer, or atomizer.

In one embodiment, the system further includes one more auxiliary component comprising salicylate-based polyanhydride-esters formulated to degrade and release salicylic acid for anti-inflammatory effect; fillers, such as glucosamine, glucosaminoglycans, and chondroitin sulfate; anti-inflammatory drugs; rapamycines and analogs, such as everolimus and biolimus; dexamethasone; M-prednisolone; interferon γ-1b; leflunomide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG; plasticizers, including cellulose and/or non-reactive PEG compounds, such as PEG-hydroxyl compounds; therapeutic agents such as stem cells, antibodies, antimicrobials, collagens, genes, DNA, and other therapeutic agents; hemostatic agents such as thrombin, chitosan, diatomaceous earth (CELOX Material), silver, and/or GELFOAM® Material; growth factors; vasoconstrictors such as Ephinephrine (which includes hydroxyl groups (—OH) and an amine group (—NH) that could be incorporated to react with PEG-SG); lydocaine; and comparable compounds.

Another example of auxiliary components that can be added include an iodinated moiety such as providone-iodine (polyvinylpyrrolidone and iodine) for antifungal/antibacterial topical application and wound healing.

Another example of auxiliary components that can be added include aloe vera, also known as the medicinal alow, which contains iodine, for wound healing.

Another example of auxiliary components that can be added include fluorocarbons (fluorine substituted hydrocarbons) and perfluorocarbons (fluorocarbons in which all of the hydrogen atoms have been replaced with fluorine), such as perfluorodecalin (CAS No. 306-94-5) and perfluorthributylamine. These compounds, because of their ability to dissolve large amounts of oxygen, can be applied topically, to provide extra oxygen to a specific location, to accelerate wound healing.

With current state of the art emulsion technology, fluoro-materials can be incorporated into the hydrogel composition. Fluoro-materials can be used to pressurize delivery units for the first and/or second components, to mix and deliver the hydrogel composition with O2 (oxygen) to the wound site.

Another representative aspect of the invention provides a method for treating burn tissue comprising (i) identifying a burn tissue site; (ii) manipulating a dispensing unit to mix a first biocompatible, synthetic, electrophilic polymer solution with a second biocompatible, synthetic, nucleophilic polymer solution to form a synthetic hydrogel composition, the first solution being essentially free of human or bovine albumin and other biological molecules and comprising poly(ethylene glycol) (PEG) Succinimidyl Glutarate having a functionality of four and a molecular weight of about 10,000 g/mole, the second solution also being essentially free of human or bovine albumin and other biological molecules and including Poly-L-Lysine hydrobromide having a number of active surface lysines of at least twenty (20) per 5000 M/W, optionally blended with a poly(ethylene glycol) (PEG) Amine having a functionality of four and a molecular weight of about 10,000 g/mole; and (iii) manipulating the dispensing unit to topically spray the synthetic hydrogel composition in situ onto the burn tissue site to provide at least one of the following treatments outcomes: use of the synthetic hydrogel composition as a hemostatic agent; and/or use of the synthetic hydrogel composition as a graft fixation agent; and/or use of the synthetic hydrogel composition to reduce need for postoperative wound care; and/or use of the synthetic hydrogel composition to reduce blood loss in an individual for whom blood transfusion is unacceptable.

Another representative aspect of the invention provides a method comprising (i) providing a first solution comprising a biocompatible, synthetic, electrophilic polymer component including a multi-arm poly(ethylene glycol) (PEG) Succinimidyl Glutarate; (ii) providing a second solution comprising a biocompatible, synthetic, nucleophilic polymer component essentially free of human or bovine albumin and other biological molecules and including a target amount of a polypeptide moiety having a number of active surface lysines of at least twenty (20) per 5000 M/W, the second solution, when mixed with the first solution, forming a synthetic hydrogel composition; (iii) titrating the target amount and molecular weight of a polypeptide moiety in the synthetic hydrogel composition to change the physical properties of the synthetic hydrogel composition in terms of elasticity; and/or stability during storage prior to use (shelf life); and/or gelation time during use; and/or degradation time after use; and (iv) instructing mixing of the first solution with the second solution in situ to form a synthetic hydrogel composition and applying the synthetic hydrogel composition onto a targeted tissue site to promote a therapeutic benefit due to the physical properties of the synthetic hydrogel composition.

In one embodiment, the polypeptide moiety is Poly-L-Lysine hydrobromide.

Another representative aspect of the invention provides a method comprising (i) providing a biocompatible, synthetic, electrophilic polymer component comprising a poly(ethylene glycol) (PEG) Succinimidyl Glutarate having a functionality of four and a molecular weight of about 10,000 g/mole; (ii) providing a biocompatible, synthetic, nucleophilic polymer component comprises a poly(ethylene glycol) (PEG) Amine having a functionality of four and a molecular weight of about 10,000 g/mole that upon mixing with the electrophilic polymer component undergoes a gelation process to form a hydrogel; and (iii) delaying onset of the gelation process by blending with the nucleophilic polymer component a Poly-L-Lysine hydrobromide having a molecular weight of greater than about 4000 g/mole.

Other features and advantages of the various aspects of the inventions are set forth in the following specification and drawings, as well as being defined in the appended claims.

### BRIEF DESCRIPTION OF THE DRAWINGS

FIG. 1 is a diagrammatic view of a system for creating families of biocompatible, synthetic compositions having diverse therapeutic indications.

FIG. 2A is a representative embodiment of a delivery system for a biocompatible, synthetic composition that embodies features of the invention, the applicator tip comprising a needle.

FIG. 2B is a representative embodiment of a delivery system for a biocompatible, synthetic composition that embodies features of the invention, the applicator tip comprising a spray tip.

FIG. 2C are representative spray tips that can be used in association with the delivery device shown in FIG. 2B.

FIG. 3 is a representative embodiment of a delivery system for a biocompatible, synthetic composition that embodies features of the invention, the applicator tip comprising an atomizing tip.

FIG. 4 is a representative embodiment of a delivery system for a biocompatible, synthetic composition that embodies features of the invention, the applicator tip being carried on the end of a catheter tube for endoscopic applications.

FIG. 5 is a representative embodiment of a delivery system for a biocompatible, synthetic composition that embodies features of the invention, the delivery system comprising a instrument that delivers components under pressure and a hand held applicator for the pressurized components.

FIGS. 6A to 6F FIG. 3 is a representative embodiment of a delivery system for a biocompatible, synthetic composition that embodies features of the invention, the delivery system comprising a self-contained hand-held device that includes sources of pressure to deliver reconstituted lyophilized components.

FIG. 7 is a graph showing the accumulation of gel strength G′ (Pascals) over time of a prepared electrophilic component mixed with a prepared nucleophilic component, as described in Example 1.

FIG. 8 is a graph showing the accumulation of gel strength G′ (Pascals) over time of a prepared electrophilic component mixed with a prepared nucleophilic component, as described in Example 2, and as compared to the mixture described in Example 1.

FIG. 9 are graphs showing the accumulating gel strength G′ (Pascals) of conventional fibrin adhesive measured over time, as described in Example 3.

FIG. 10 are graphs that compare the accumulation of gel strength G′ (Pascals) of conventional fibrin adhesive to the accumulation of gel strength G′ (Pascals) of the PEG-SG and Poly-L-Lysine Hydrobromide composition of Example 1, as described in Example 3.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

### I. System Overview

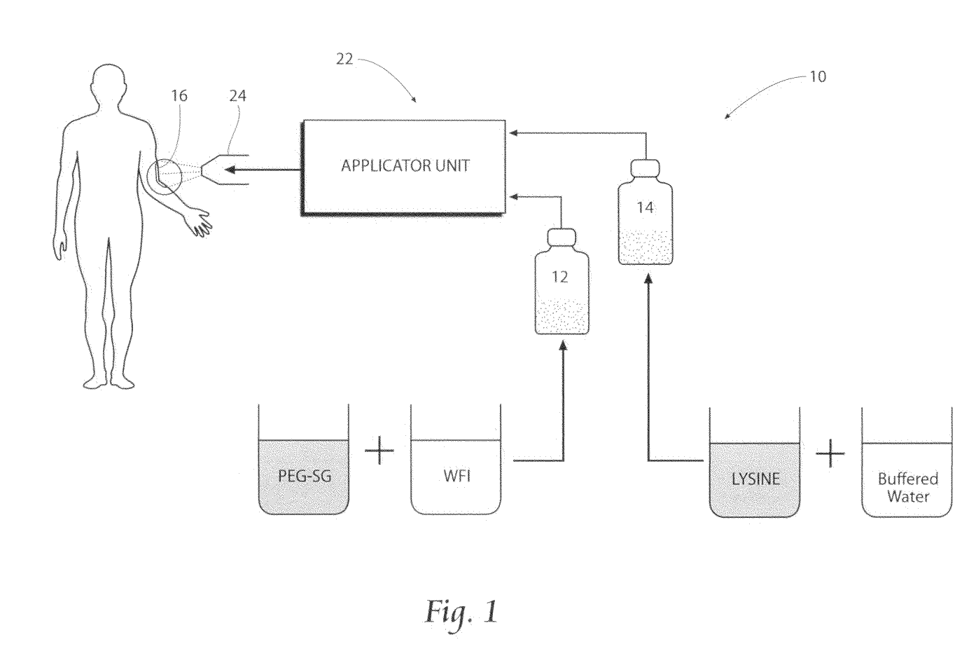

FIG. 1 shows a system 10 for creating families of biocompatible, synthetic compositions having diverse therapeutic indications. The genus platform for the system 10 includes a biocompatible, synthetic electrophilic polymer component 12 that includes a poly(ethylene glycol) (PEG) that is mixed with a biocompatible, synthetic nucleophilic component 14 that includes poly-L-Lysine hydrobromide. The components 12 and 14 are solids that are placed in solution for delivery.

The two components 12 and 14, when mixed in a liquid state, are reactive. When mixed, the two components 12 and 14 react by cross-linking, forming a solid matrix composition 16, or hydrogel. By “cross-linking,” it is meant that the hydrogel composition contains intermolecular crosslinks and optionally intramolecular crosslinks as well, arising from the formation of covalent bonds.

Depending upon the characteristics of the two components 12 and 14 selected, different species of matrix compositions 16 can be formed. These different species lend themselves to use in diverse therapeutic indications. The therapeutic indications for compositions that incorporate one or more aspects of the invention include: (i) collagen restoration/replacement (e.g., topical application or void filling by injection to fill wrinkles, or for biopsy sealing); (ii) drug delivery (e.g., the delivery of glucosamine and chondroitin sulfate into the spine area or other body regions); (iii) stem cell or growth factor delivery (e.g., the delivery of stem cells and/or growth factors into the spine area or other body regions); (iv) tissue sealants/adhesives; (v) wound healing and the control of bleeding or fluid leakage in body tissue (e.g., lung sealing, liver lacerations, or hemostasis); (vi) tissue, muscle, and bone growth and regeneration; (vii) dermatology (e.g., topical cosmetic and therapeutic creams, shampoos, soaps, and oils); (vii) internal and external bonding and coating of tissue and instruments, e.g., coatings for burn victims, artificial skin, adhesion prevention, coatings on polymers, or coatings for implant devices such as, e.g., stents; (viii) vascular grafts; and (ix) burn operations.

In use, the synthetic hydrogel does not evoke swelling, fragmentation, embolization, or the coagulation cascade. The synthetic hydrogel can be delivered to otherwise hard-to-reach sites, e.g., by endoscopy. The synthetic hydrogel will not interfere with the intended effect of other adhesives or therapeutic compositions used in combination with the synthetic hydrogel, e.g., an adhesive that is applied to a prosthesis to fixate the prosthesis to bone or another tissue site. The synthetic hydrogel aids in wound healing and hemostasis with less scarring.

### A. ELECTROPHILIC COMPONENT

In a representative embodiment, the biocompatible, synthetic, electrophilic polymer component comprises a multi-arm poly(ethylene glycol) (PEG) Succinimidyl Glutarate having a functionality of at least three and preferably four—or, in short hand, 4-Arm PEG Succinimidyl Glutarate (PEG-SG)—having a molecular weight of about 10,000 g/mole (available from Polymer Source, Inc. at www.polymersource.com).

The PEG-SG is dissolved in Sterile Water for Injection USP (SWI) (available from Abbott Laboratories) for delivery. In a representative embodiment, a targeted weight of 0.25 g of PEG-SG is added to a targeted volume of 1.25 cc of Sterile Water for Injection USP and mixed. No buffering material need be added. One (1) cc of the resulting HPLC Water/PEG-SG solution is housed in a sterile dispensing container. An applicator unit receives the PEG-SG solution for dispensing during use, as will be described in greater detail later.

### B. THE NUCLEOPHILIC COMPONENT

In a representative embodiment, the nucleophilic component 14 includes a Poly-L-Lysine hydrobromide (in shorthand Poly-L-HBr) having a molecular weight of at least 4 g/mole, with an upper limit dependent upon the physical properties desired of, e.g., 4000 g/mole, or greater than 4000 (e.g., 15,000 g/mole), and also greater than 70,000 (available from ICN Biomedicals, Inc. at www.mpbio.com). The Poly-L-Lysine hydrobromide is dissolved in buffered HPLC-grade water for delivery.

Poly-L-Lysine hydrobromide is not characterized in terms of “functionality” as are PEG materials (i.e., 4-Arm PEG means a PEG with a functionality of four). Poly-L-Lysine hydrobromide is a polypeptide moiety (like albumin) that is characterized not by “functionality” but by reference to the number of active surface lysines, which for Poly-L-Lysine hydrobromide is at least twenty (20) per 5000 M/W.

The Poly-L-Lysine hydrobromide molecule is relatively long and thereby provides flexibility to the solid matrix hydrogel composition 16. The Poly-L-Lysine hydrobromide molecule takes the place of human or bovine albumin and other biological molecules, which can have undesired after-effects. By titrating the amount and molecular weight of Poly-L-Lysine hydrobromide in the composition, one can change the physical properties of the resulting hydrogel composition in terms of, e.g., elasticity; stability during storage prior to use (shelf life); gelation time during use; and degradation time after use.

If desired, the nucleophilic component 14 can include a blend of Poly-L-Lysine hydrobromide, as just described, and a multi-arm poly(ethylene glycol) (PEG) Amine having a functionality of at least three and preferably four—or, in short hand, 4-Arm PEG Amine—having a molecular weight of about 10,000 g/mole (available from Polymer Source, Inc. at www.polymersource.com). The PEG-Amine and Poly-L-Lysine hydrobromide are dissolved in HPLC-grade water for delivery.

In a representative blended embodiment, a targeted weight of 0.03 g of PEG-Amine and a target weight of 0.200 g of the Poly-L-Lysine hydrobromide are added to a target volume of 1.25 cc of HPLC-grade water (pH 9.7, with a buffer material such as tris (hydroxymethyl) aminomethane buffer) and mixed. One (1) cc of the HPLC Water/PEG-Amine/Poly-L-Lysine hydrobromide solution is housed in a second sterile container. The dispensing unit receives the contents of second container along with the contents of the first container for mixing and dispensing during use, as will be described in greater detail later.

Kits may be provided to facilitate mixing of the electrophilic and nucleophilic components 12 and 14 on site at the instant of use. The kits may include instructions for use, which direct the use of the composition for targeted therapeutic indications.

In a representative composition, the ratio of the nucleophilic component 14 to the electrophilic component 12 is selected to be about 0.7 to 1.0. This ratio assures that there will be a greater amount of SG functional groups than amine functional groups. This selected ratio provides that substantially all amine functional groups will be reacted with the SG functional groups during the cross-linking process. It is believed that the substantial absence of unreacted amine functional groups enhances the overall biocompatibility of the resulting hydrogel.

The two components 12 and 14, when mixed in a liquid state, are reactive. When mixed, the two components 12 and 14 react by cross-linking, forming a solid matrix composition 16, or hydrogel. Depending upon the characteristics of the two components 12 and 14 selected, different species of matrix compositions 16 can be formed. These different species lend themselves to use in diverse therapeutic indications, as described above.

The formed hydrogel can comprise a foam containing the two active components 12 and 14. In this arrangement, the first component (PEG-SG) can incorporate a sodium bicarbonate, and the second component 14 (Poly-L-HBr, with or without PEG-Amine) can incorporate an acetic acid that reacts with the sodium bicarbonate by foaming.

### C. THE DELIVERY SYSTEM

The delivery system for the components 12 and 14 can be variously constructed.

As shown in FIG. 1, the electrophilic component 12 and the nucleophilic component 14 can be separately prepared and housed in separate dispensing containers or vials, as previously described. For example, the PEG-SG can be placed in solution with water for injection (WFI) and contained in a first sterile vial. The Poly-L-Lysine Hydrobromide can also be placed in solution with buffered water (with PEG-Amine, if desired) and contained in a second sterile vial.

Alternatively, the PEG-SG, Poly-L-Lysine Hydrobromide, and (if desired) PEG-Amine can be lyophilized for fast mixing. In this arrangement, lyophilized component or components can take the form of microliter aliquots of solution that are lyophilized as precise and durable units of use spheres packaged inside vials or other delivery devices. An example of technology that can place PEG and Poly-L-Lysine Hydrobromide into lyophilized spheres for delivery can be found at www.biolph.com. During the lyophilization process, desired buffers can also be added, so that, at the instance of use, all that is required is sterile water.

Of course, there are various packaging options for the vials and their contents.

For example, one packaging option comprises four vials (Vial 1: PEG-SG; Vial 2: WFI, for use with PEG-SG in Vial 1; Vial 3: Poly-L-HBr, with or without PEG-Amine; and Vial 4: Buffered HPLC water, for use with the contents of Vial 3).

For example, another packaging option comprises three vials (Vial 1: PEG-SG; Vial 2: WFI for use with Vial 1 and Vial 3; Vial 3: Poly-L-HBr, with or without PEG-Amine, and Buffer).

For example, another packaging option comprises two vials (Vial 1: PEG-SG; Vial 2 Poly-L-HBr, with or without PEG-Amine, and Buffer; and the hospital provides WFI for Vials 1 and 2).

At the instance of use, the first and second vials are placed in a dispensing apparatus, or applicator unit 22, which is desirably disposable. The dispensing unit 22 mixes the electrophilic component 12 and the nucleophilic component 14 and dispenses the mixture in situ. The dispensing unit 22 can include a static mixing element (or it may not include a static mixing element), and an appropriate delivery tip 24. As will be described in greater detail, the dispensing unit 22 can, depending upon the dispensing environment, be sized and configured as an integrated hand held device, or as an integrated hand held endoscopic device, or as an instrument system having a dispensing unit and a mixing unit. The form, fit, and function of the dispensing unit can be optimized to match the specific requirements of the targeted indication. The dispensing tip can also be sized and configured as a needle, sprayer, or atomizer.

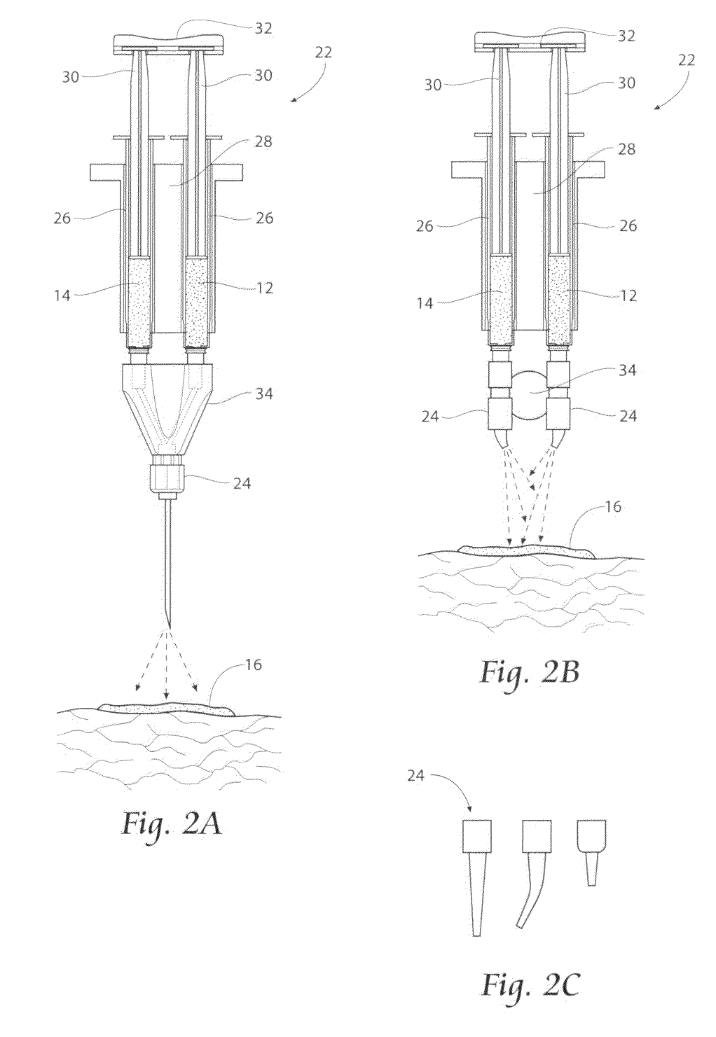

For example (as shown in FIGS. 2A and 2B), the dispensing unit 22 can comprise a pair of manual syringes barrels 26 joined by a clip 28, each having a plunger 30 that are mutually joined by a joiner clip 32. The vials of the electrophilic component 12 and the nucleophilic component 14 are loaded into the syringe barrels 26. A applicator joining piece 34 includes the delivery tip 24. Manual advancement of the plungers 30 by the caregiver conveys the electrophilic component 12 and the nucleophilic component 14 from their respective vials for mixing in the joining piece 34 and for dispensing onto the targeted tissue site through the delivery tip 24. The delivery tip 24 can comprise, e.g., a needle 36 (FIG. 2A) or a spray nozzle 38 (FIG. 2B). As FIG. 2C shows, the spray nozzle 38 can take various different forms, depending upon the manner that the hydrogel material 16 is to be applied.

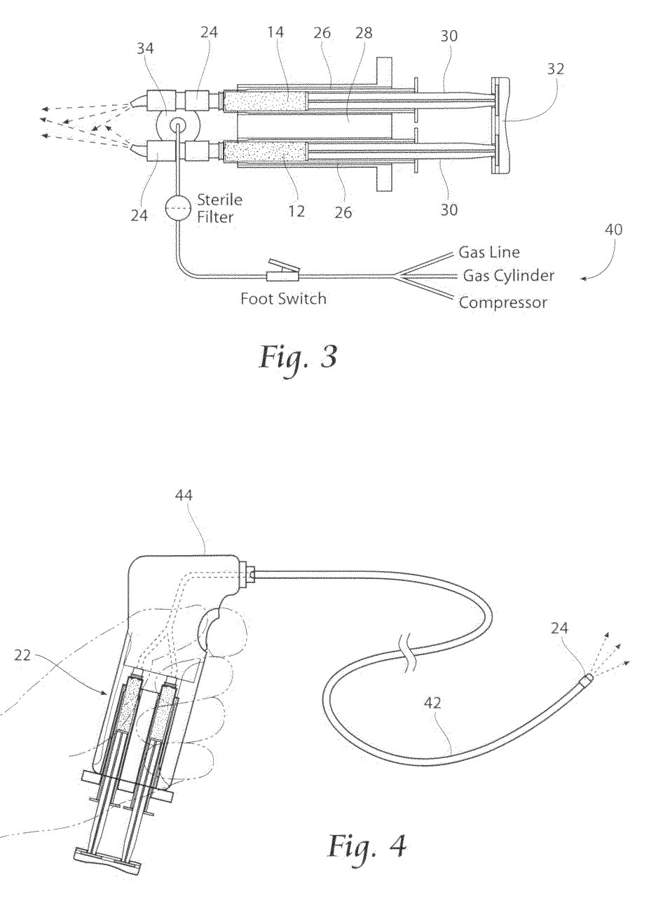

In an alternative arrangement (as shown in FIG. 3), a pressure source 40 such as a gas line, gas cylinder, or compressor can be coupled to the applicator joiner 34 via a foot switch control to introduce air into the mixed electrophilic component 12 and the nucleophilic component 14 as they are being mixed and dispensed from the applicator tip 24. The gas serves to atomize the mixture 16 during application.

In another alternative arrangement (as shown in FIG. 4), the applicator tip can be carried at the end of a catheter tube coupled to the applicator unit 22 contained in a proximal housing 44 in to provide for endoscopic spraying of the mixture to the targeted tissue site.

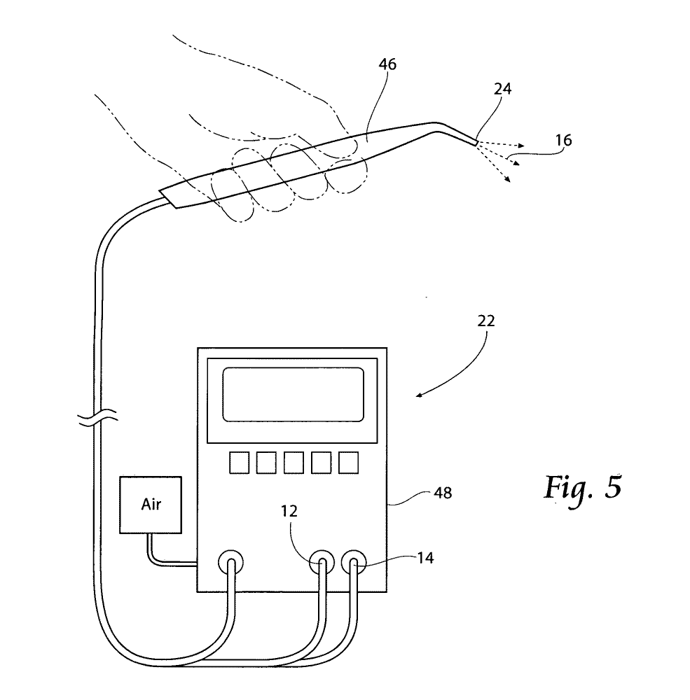

In another environment (as shown in FIG. 5), the applicator unit 22 can comprise a system having a hand held dispensing unit 46 and a remote pumping instrument 48. The pumping instrument 48 receives the electrophilic component 12, the nucleophilic component 14, and pressurized air for mixing and conveyance to the dispensing unit 46. As illustrated, the dispensing unit 46 comprises a pen-shaped device that sprays the mixture of the electrophilic component 12 and the nucleophilic component 14 onto the targeted tissue site. An example of this type of system can be found at www.vivostat.com. This arrangement allows for constant spraying over a larger tissue surface area, as well as accommodates pin point applications, such as micro-anastomosis and other difficult-to-reach areas.

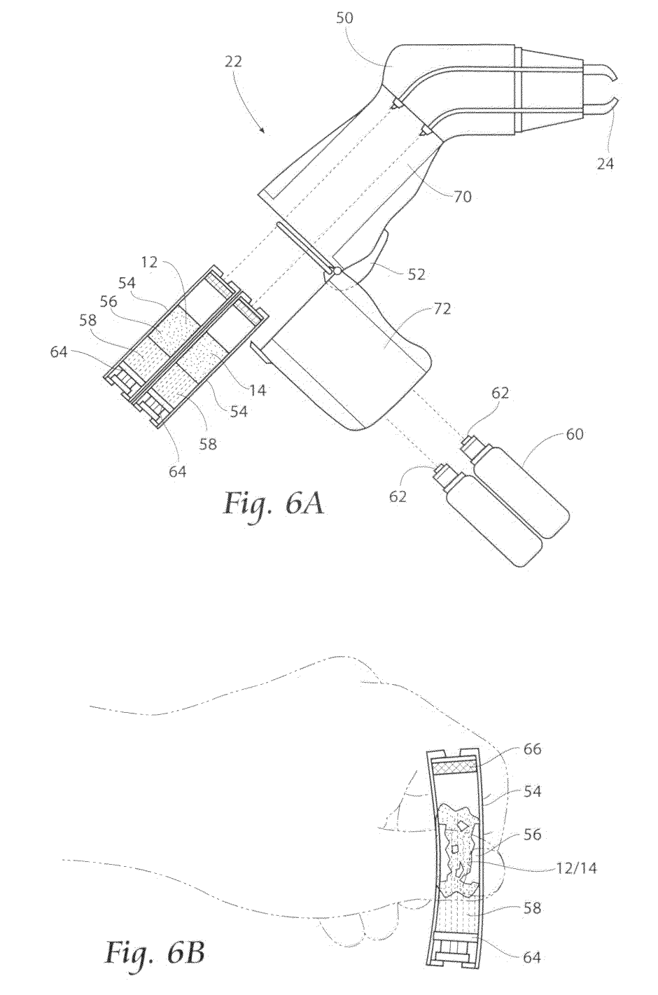

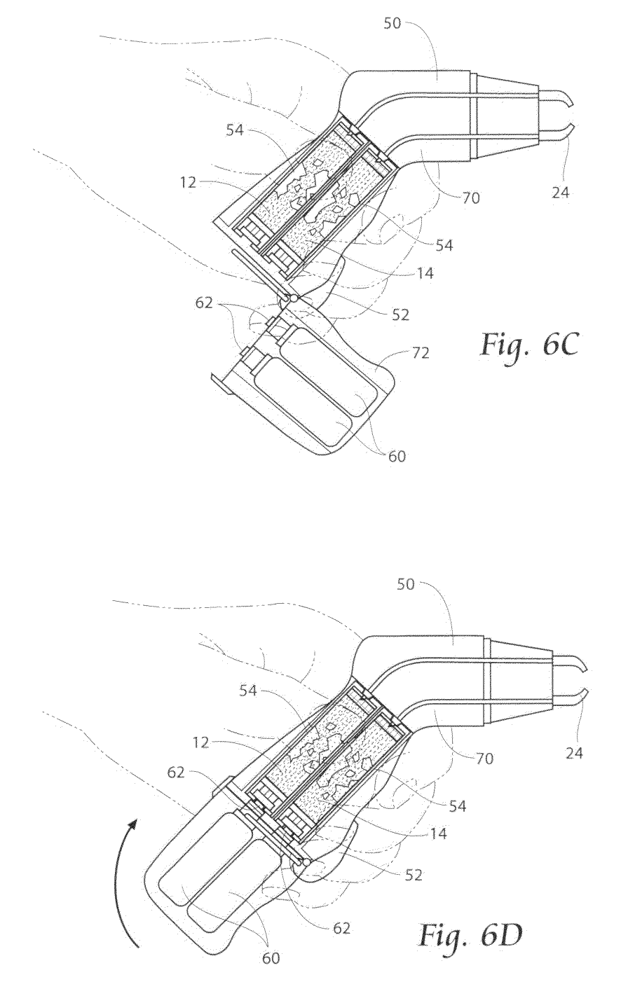

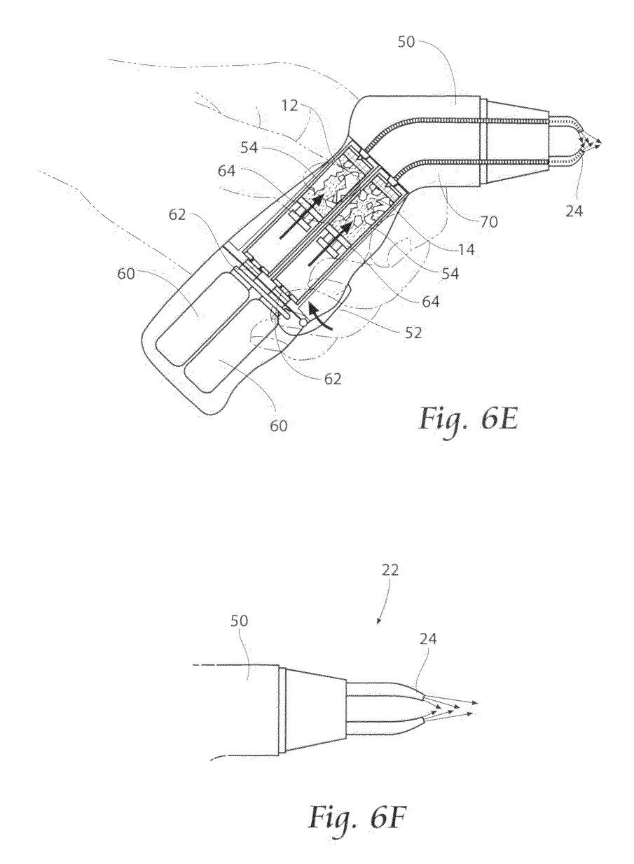

In another environment (as shown in FIG. 6A), the applicator unit 22 comprises a fully-disposable, self-contained hand-held device 50 that leverages the chemical properties of the compound to enhance usability. The device 50 includes a handle 52 that pivots open into upper and lower section 70 and 72. The upper section 70 receives single use, sterile component cartridges 54. The lower section 72 receives pressurized air cartridges 60 with valves 62.

As FIG. 6A shows, each component cartridge 54 contains lyophilized particles of the respective component 12/14 enclosed within a frangible compartment 56 next to a compartment 58 of sterile water. This packaging enables the component 12/14 to be conveniently stored in lyophilized form at room temperature.

As FIG. 6B shows, prior to use, each frangible compartment 56 is broken, e.g., by pinching or bending the cartridge 54, to allow the sterile water to mix with and reconstitute the lyophilized component 12/14 within the cartridge 54. As FIG. 6C shows, the cartridges 54 can then be inserted into the upper section 70 (which is pivoted open), where their distal ends couple with channels that lead to the applicator tip 24.

As FIG. 6C shows, the pressurized air cartridges 60 can also be loaded into the lower section 72. The upper and lower sections 70 and 72 of the handle can then be closed, as FIG. 6D shows. The air canisters 60, when loaded, are coupled by valves 62 to the cartridges 54. The trigger 52 opens the valves 62, to allow the pressurized air to advance pistons 64 through the cartridges 54 and convey the reconstituted components 12 and 14 to the applicator tip 24, as shown in FIG. 6E. A filter 66 at the distal end of each cartridge 54 traps remnants of the frangible compartment 56 within the cartridge 54. The applicator tip 24 comprises a dual-outlet nozzle (see FIG. 6E) that automatically mixes the two reactive components 12 and 14 after they have exited the applicator tip 24, so there is no pre-mixing and the tip does not clog. FIG. 6F shows an alternative form for a dual-outlet nozzle that mixes components outside the nozzle.

A single trigger 52 can open the valves concurrently for simultaneously delivery of the components through the dual-outlet nozzle. Alternatively, dual triggers can open the valves independently. In the latter arrangement, the components 12 and 14 can be conveyed simultaneously for mixing through both spray nozzles (by operating both triggers simultaneously), or the applicator tip 24 can be intermittently cleared or purged by the delivery of an aliquot of one then the other of the components 12 and 14 through a respective one of the outlets of the dual-outlet spay nozzle (by operating the triggers independently).

The two pressurized canisters 60 contained within the device 50 allow the device 50 to spray the composition 16 over the desired area without the aid of an external pressurized air instrument or other equipment that can be cumbersome, confusing, and expensive. With the touch of the trigger 52, the physician can spray the synthetic hydrogel composition 16 described herein consistently over diffuse or hard to reach surface areas.

### Example 1

### PEG-SG and Poly-L-Lysine Hydrobromide

Preparation of the electrophilic component: A weight of 0.25 g of 4-Arm PEG-SG (M/W 10,000 g/mole) is added to a volume of 1.25 cc of water for injection (WFI), and mixed. No buffering material is added. One (1) cc of the resulting WFI/PEG-SG solution is housed in a sterile dispensing syringe.

Preparation of the nucleophilic component: A weight of 0.20 g of Poly-L-Lysine hydrobromide (M/W of about 30,000 to greater than 70,000 g/mole) are added to a volume of 1.25 cc of HPLC-grade water (pH 9.73, with tris (hydroxymethyl)aminomethane buffer material), and mixed. One (1) cc of the HPLC Water/Poly-L-Lysine hydrobromide solution is housed in a sterile dispensing syringe.

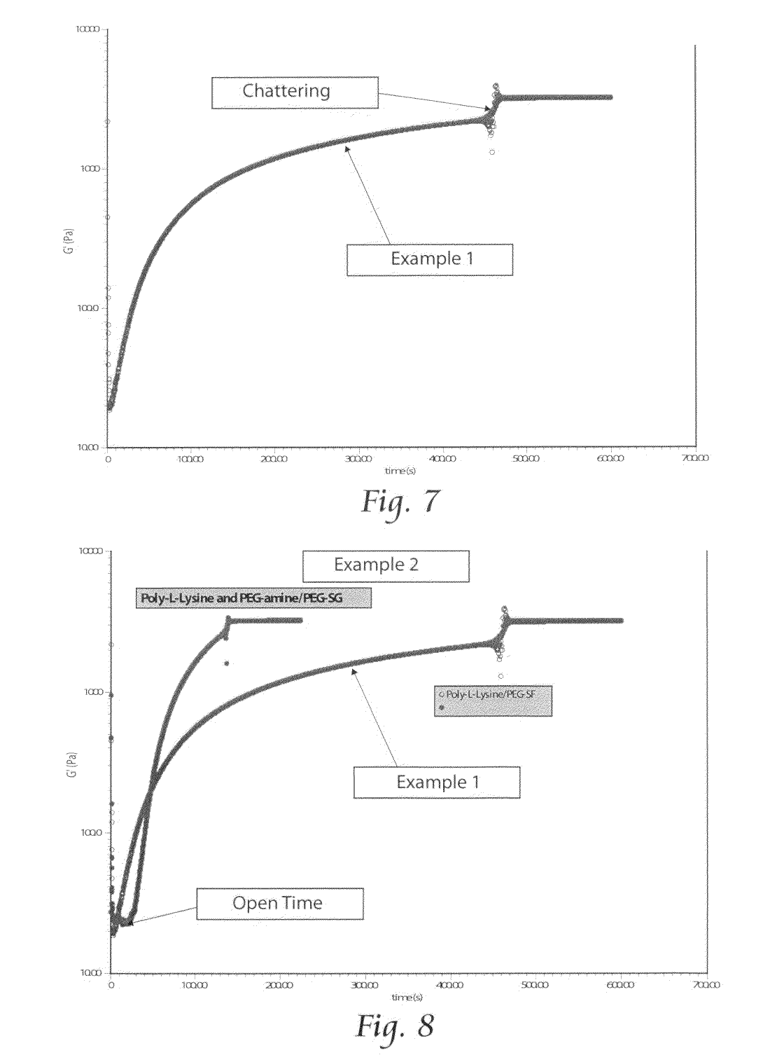

Mixing of the components/gelation: A volume of 1 cc of the prepared electrophilic component is mixed with a volume of 1 cc of the prepared nucleophilic component (total mixed volume=2 cc). The accumulating gel strength G′ (Pascals) of the mixture over time is measured on an AR2000EX Rheometer (2% strain, in oscillation mode frequency 1 Hz fast oscillation mode, 10 data points per second, time sweep, 25 mm plate, 1.5 mm gap, at 25-degrees C.). The resulting graph of G′ (Pascels) over time is shown in FIG. 7.

The graph in FIG. 7 shows an increase in gel strength over time. The “chattering” observed at 442 seconds (2196 Pa) demonstrates excellent adhesive properties and cohesive properties in a time period well suited for treating wounds and achieving hemostasis, such as in connection with burn operations, or the coverage of large wound sites like the skin, liver, or lung sealing bleeding sites.

### Example 2

### PEG-SG and Blend of PEG-Amine and Poly-L-Lysine Hydrobromide

Preparation of the electrophilic component: A weight of 0.25 g of 4-Arm PEG-SG (M/W 10,000 g/mole) is added to a volume of 1.25 cc of water for injection (WFI), and mixed. No buffering material is added. One (1) cc of the resulting WFI/PEG-SG solution is housed in a sterile dispensing syringe.

Preparation of the nucleophilic component: A weight of 0.13 g of PEG-Amine (M/W 10,000 g/mole) and a weight of 0.03 g of the Poly-L-Lysine hydrobromide (M/W 4,000 to 15,000 g/mole) are added to a volume of 1.25 cc of HPLC-grade water (pH 9.72, with tris (hydroxymethyl) aminomethane buffer material), and mixed. One (1) cc of the HPLC Water/PEG-Amine/Poly-L-Lysine hydrobromide solution is housed in a sterile dispensing syringe.

Mixing of the components/gelation: A volume of 1 cc of the prepared electrophilic component 12 is mixed with a volume of 1 cc of the prepared nucleophilic component (total mixed volume=2 cc). The accumulating gel strength G′ (Pascals) of the mixture over time is measured on an AR2000EX Rheometer (2% strain, in oscillation mode frequency 1 Hz fast oscillation mode, 10 data points per second, time sweep, 25 mm plate, 1.5 mm gap, at 25-degrees C.). The resulting graph of G′ (Pascels) over time is shown in FIG. 8, with Example 1, for comparison.

The graph shown in FIG. 8 shows a more rapid increase in gel strength over time when nucleophilic component includes a blend of PEG-Amine and Poly-L-Lysine Hydrobromide (Example 2). The “chattering” observed at about 140 seconds (2200 Pa) demonstrates excellent adhesive properties and cohesive properties in a time period well suited for treating wounds and achieving hemostasis, such as in connection with burn operations, or the coverage of large wound sites like the skin, liver, or lung sealing bleeding sites.

The graph of FIG. 8 also shows that the blend of PEG-Amine and Poly-L-Lysine Hydrobromide (Example 2) exhibits a delay in gelation for about 25 seconds after mixing, which is called “open time.” During this open time, viscosity does not change. The “open time” is beneficial in environments that require passage of the two components 12 and 14 through the lumen of a delivery device without gelation (e.g., in neurological or laparoscopic environments). Passage of the components 12 and 14 can therefore occur without clogging the lumen of a delivery device. Gelation occurs later, after the components 12 and 14 have exited the delivery device 16 and reside proximal to the targeted treatment site.

### Example 3

### Comparison to Conventional Fibrin Adhesive

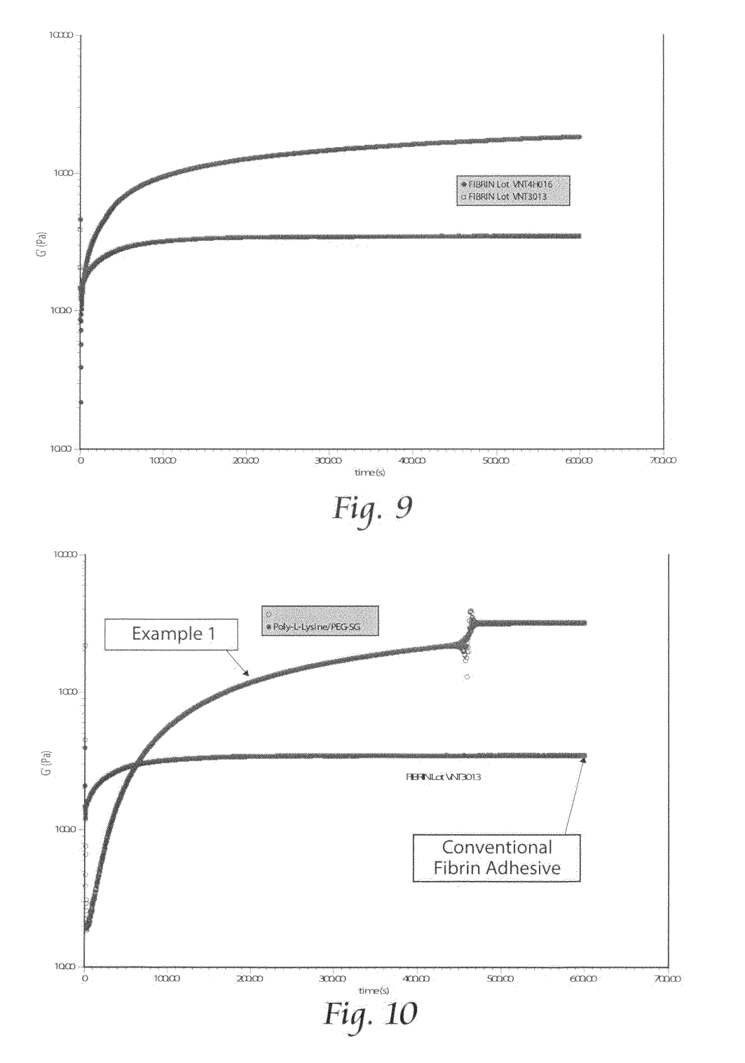

The graphs shown in FIG. 9 show the accumulating gel strength G′ (Pascals) of conventional fibrin adhesive (Baxter Healthcare Corporation) measured over time on an AR2000EX Rheometer (2% strain, in oscillation mode frequency 1 Hz fast oscillation mode, 10 data points per second, time sweep, 25 mm plate, 1.5 mm gap, at 25-degrees C.).

The graphs shown in FIG. 10 compare the accumulation of gel strength G′ (Pascals) of conventional fibrin adhesive (Baxter Healthcare Corporation) to the accumulation of gel strength G′ (Pascals) of the PEG-SG and Poly-L-Lysine Hydrobromide composition of Example 1. The graph of FIG. 10 shows that the composition of Example 1 has adhesive properties and cohesive properties superior to conventional fibrin adhesives.

### D. ADDITIVE COMPONENTS

The synthetic hydrogel composition may also incorporate one or more auxiliary components that impart other mechanical and/or therapeutic benefits.

For example, fast-degrading, salicylate-based polyanhydride-esters) can be incorporated to degrade and release the active component (salicylic acid, or aspirin) for anti-inflammatory effect.

Other auxiliary components that can be added include fillers, such as glucosamine, glucosaminoglycans, and chondroitin sulfate; anti-inflamatory drugs; rapamycines and analogs, such as everolimus and biolimus or of the kind used on drug-eluting stents by Biosensors International (see. E.g., Prospectus, Biosensors International, Apr. 22, 2005, Registered with the Monetary Authority of Singapore on Apr. 22, 2005); dexamethasone; M-prednisolone; interferon γ-1b; leflunomide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG; plasticizers, including cellulose and/or non-reactive PEG compounds, such as PEG-hydroxyl compounds; therapeutic agents such as stem cells, antibodies, antimicrobials, collagens, genes, DNA, and other therapeutic agents; hemostatic agents such as thrombin, chitosan, diatomaceous earth (CELOX Material), silver, and/or GELFOAM® Material; growth factors; vasoconstrictors such as Ephinephrine (which includes hydroxyl groups (—OH) and an amine group (—NH) that could be incorporated to react with PEG-SG); lydocaine; and comparable compounds.

Another example of auxiliary components that can be added include an iodinated salicylic acid (ISA) molecule (including but not limited to 5-iodosalicylic acid and 3,5-diiodosalicylic acid) incorporated into a polyanhydride-ester formulated to degrade and release salicylic acid for anti-inflammatory effect and release iodine for therapeutic infection prevention and wound healing.

Another example of auxiliary components that can be added include an iodinated moiety such as providone-iodine (polyvinylpyrrolidone and iodine) for antifungal/antibacterial topical application and wound healing.

Another example of auxiliary components that can be added include aloe vera, also known as the medicinal alow, which contains iodine, for wound healing.

Another example of auxiliary components that can be added include fluorocarbons (fluorine substituted hydrocarbons) and perfluorocarbons (fluorocarbons in which all of the hydrogen atoms have been replaced with fluorine), such as perfluorodecalin (CAS No. 306-94-5) and perfluorthributylamine. These compounds, because of their ability to dissolve large amounts of oxygen, can be applied topically, to provide extra oxygen to a specific location, to accelerate wound healing.

With current state of the art emulsion technology, fluoro-materials can be incorporated into the hydrogel composition. Fluoro-materials can be used to pressurize delivery units for the first and/or second components, to mix and deliver the hydrogel composition with O2 (oxygen) to the wound site.

The auxiliary components may be added to either the nucleophilic or the electrophilic components 12 and 14, and could also be added to the components 12 and 14 prior to or concurrent with delivery of the components 12 and 14 to the targeted application site.

### E. ILLUSTRATIVE USE IN BURN OPERATIONS

The properties of the synthetic hydrogel compositions described herein make possible dramatic improvements in outcomes and reducing the burden of healthcare costs as it pertains to patients undergoing skin grafting following severe burns.

The nature of burn operations can be dreadful. Surgeons use a sharp device to remove the skin from the entire area of the patient's body that has been burned; bleeding signals that the tissue is healthy and can be suitable for grafting. Next, skin is harvested from a healthy, unburned area (if available) of the patient's body using depth-controlled razor-like device called a dermatome; similarly, bleeding signals that a full thickness graft has been taken. Bleeding from a donor site is diffuse, punctuate, and profuse. Bleeding from a re-use donor is even more so. Because blood loss will be substantial, hemostasis at the donor site should be controlled before pursuing wound excision. The ideal situation is the use of two teams, one whose role is to obtain skin grafts and maintain hemostasis. The donor skin is placed over the burn site and stapled into place. The wounds are covered after each excision, possibly with an epinephrine and thrombin soaked cloth. The result of this operation is excessive blood loss, and if the graft survives, then it is considered a successful burn treatment surgery. For an average patient, with 25% of their body surface area removed (which is an area slightly larger than the patient's arm), the blood loss amounts to approximately 4.5 units of blood, even if completed over multiple surgeries. As a result, hemorrhage control and the need for transfusion is a major medical concern.

Tourniquets and epinephrine-soaked sheets have been proven ineffective at controlling intraoperative blood loss. Additionally, pooling of blood or other fluids under the graft at the burn site is common, and can result in graft failure and poor cosmetic results. Fibrin sealants have been investigated for the purpose of limiting blood loss with the anticipated impact of minimizing blood loss, reducing operative time, and increasing graft success rate. Unfortunately, they leave much to be desired: the product itself has poor adhesive properties, the syringe-based applicators are difficult to use (they clog and the reaction time is too quick to dispense the product as desired), they are very expensive, and carry the risk of disease transmission.

The synthetic hydrogel compositions described herein function as special purpose intraoperative and dermal adhesive compositions, providing safe, effective, and resource efficient perioperative and trauma-related tissue repair, particularly where excessive bleeding and/or impaired coagulation precludes the use of conventional modalities. Due to their excellent adhesive properties and cohesive properties, as well as the purposeful, predictable manner in which gel strength accumulates, the synthetic hydrogel compositions described herein address the aforementioned shortcomings of existing products. The synthetic hydrogel compositions described herein are well suited for use as hemostatic agents in the early excision of a large burn or an extremity burn. The synthetic hydrogel compositions described herein are useful as fixation agents in virtually all cases, especially if a sheet graft is planned or if the graft site involves crucial areas or particularly cosmetically important areas like the face or the hands, where any degree of graft loss results in unacceptable cosmetic deformity. The synthetic hydrogel compositions described herein also offer critical advantages in pediatric cases, particularly in the reduced need for postoperative wound care. For those for whom blood transfusion is unacceptable, the employment of the synthetic hydrogel compositions described herein to reduce blood loss can literally make the difference between life and death. Further, the synthetic hydrogel compositions described herein lack the complexity of preparation and application encountered with conventional fibrin adhesives.

### F. OTHER BENEFICIAL PROPERTIES/INDICATIONS

The synthetic hydrogel compositions described herein are elastic, serve as an effective sealant on wet and blood spotted tissues, and can be effectively applied as a spray and used alone or in combination with other solid matricies.

In a porcine lung resection, the synthetic hydrogel compositions described herein served as an effective elastic sealant and could be applied as a spray. When applied as a spray, the synthetic hydrogel compositions described herein create an air-tight seal over a porous tissue and expands and contracts while maintaining adhesion.

Using a liver laceration model, the synthetic hydrogel compositions described herein created a seal in bloody environments. The synthetic hydrogel compositions described herein adhered to wet liver tissue, sealed over a pocket of blood from the laceration that had spread over the liver surface, and maintained a seal around the blood pocket. A second liver study demonstrated that the synthetic hydrogel compositions described herein could be used in combination with a solid material. Placing GELFOAM® Material directly over the laceration, and the synthetic hydrogel compositions described herein over the solid matrix, it was demonstrated that the synthetic hydrogel compositions described herein can adhere the solid matrix to the tissue and seal the solid matrix.

The synthetic hydrogel compositions described herein create a mechanical bond with PTFE and Dacron graft materials. Covalent bonds cannot occur because of the non-reactive surfaces designed into these materials. However, when the synthetic hydrogel compositions described herein are first applied, they are able to partially penetrate the nooks and crannies of the irregular graft surfaces. Within seconds of application the synthetic hydrogel compositions described herein partially penetrate the natural holes found in the graft, and the synthetic hydrogel compositions described herein begin to set, thereby effectively molding themselves to the graft.

### G. CONCLUSION

The synthetic hydrogel compositions described herein provide diverse benefits when compared to existing technologies in this field. These benefits include (i) the synthetic hydrogel compositions described herein do not rely on a functioning coagulation cascade; (ii) the synthetic hydrogel compositions described herein provide superior adhesion properties in “wet” tissue; (iii) the synthetic hydrogel compositions described herein make possible the use of a high-tech applicator: with a single-handed use, no pre-mixing, with delivery to large areas, which is easier to direct and control, and with interchangeable designs for anti-clog tips; (iv) the synthetic hydrogel compositions described herein can provide delayed activation (the open time); (v) the synthetic hydrogel compositions described herein can provide consistency among lots, longer shelf-life at room-temperature storage, at an expensive than higher priced sealants; (vi) the synthetic hydrogel compositions described herein make possible variations in chemical formulation for target indications (e.g. aid in wound healing, minimized swelling); (vii) the molecular structure of Poly-L-HBr that the synthetic hydrogel compositions described herein includes provides greater elasticity; and (viii) the synthetic hydrogel compositions described herein provide no risk of adverse reactions to thrombin and biologic molecules.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention.

I claim:

1. A system for promoting wound healing comprising
a first solution comprising a biocompatible, synthetic, electrophilic polymer component including a multi-arm poly(ethylene glycol) (PEG) Succinimidyl Glutarate,
a second solution comprising a biocompatible, synthetic, nucleophilic polymer component essentially free of human or bovine albumin and other biological molecules and including a polypeptide moiety having a number of active surface lysines of at least twenty (20) per 5000 M/W, and optionally blended with a multi-arm poly(ethylene glycol) (PEG) Amine, and
instructions for use comprising
mixing the first and second solutions to form a synthetic hydrogel composition, and
applying the synthetic hydrogel composition by topically spraying the synthetic hydrogel composition onto a targeted wound site to promote wound healing.

2. A system according to claim 1
wherein the polypeptide moiety comprises Poly-L-Lysine hydrobromide.

3. A system according to claim 1
wherein the multi-arm poly(ethylene glycol) (PEG) Succinimidyl Glutarate has a functionality of four.

4. A system according to claim 1
wherein the optional multi-arm poly(ethylene glycol) (PEG) Amine (PEG-Amine) has a functionality of four.

5. A system according to claim 1
further including one more auxiliary component comprising salicylate-based polyanhydride-esters formulated to degrade and release salicylic acid for anti-inflammatory effect; fillers, such as glucosamine, glucosaminoglycans, and chondroitin sulfate; anti-inflamatory drugs; rapamycines and analogs, such as everohtnus and biolimus; dexamethasone; M-prednisolone; interferon γ-lb; leflunomide; mycophenolic acid; mizoribine; cyclosporine; tranilast; biorest; tacrolimus; taxius; pacitaxel; or taxol; botox; lydicane; Retin A Compound; glucosarnine; chondroitin sulfate; or Geldanamycin analogs 17-AAG or 17-DMAG; plasticizers, including cellulose and/or non-reactive PEG compounds, such as PEG-hydroxyl compounds; therapeutic agents such as stem cells, antibodies, antimicrobials, collagens, genes, DNA, and other therapeutic agents; hemostatic agents such as thrombin, chitosan, diatomaceous earth (CELOX Material), silver, and/or GELFOAM® Material; growth factors; vasoconstrictors such as Ephinephrine (which includes hydroxyl groups (—OH) and an amine group (—NH) that could be incorporated to react with PEG-SG); lydocaine; and comparable compounds.

6. A system according to claim 1
further including a dispensing unit that mixes the first and second solution and dispenses the mixture in situ through a dispensing tip, and
wherein the instructions for use direct use of the dispensing unit.

7. A system according to claim 6
wherein the dispensing unit is sized and configured as an integrated hand held device, or as an integrated hand held endoscopic device, or as an instrument system having mixing and dispensing units.

8. A system according to claim 6
wherein the dispensing tip is sized and configured as a needle, sprayer, or atomizer.

9. A method for promoting wound healing comprising
providing a first solution comprising a biocompatible, synthetic, electrophilic polymer component including a multi-arm poly(ethylene glycol) (PEG) Succinimidyl Glutarate,
providing a second solution comprising a biocompatible, synthetic, nucleophilic polymer component essentially free of human or bovine albumin and other biological molecules and including a polypeptide moiety having a number of active surface lysines of at least twenty (20) per 5000 M/W, and optionally blended with a multi-arm poly(ethylene glycol) (PEG) Amine (PEG-Amine),
mixing the first and second solutions to form a synthetic hydrogel composition, and
applying the synthetic hydrogel composition by topically spraying the synthetic hydrogel composition onto the wound site to promote wound healing.

10. A system for wound healing comprising
a first solution comprising a biocompatible, synthetic, electrophilic polymer component including a poly(ethylene glycol) (PEG) Succinimidyl Glutarate having a functionality of four and a molecular weight of about 10,000 g/mole,
a second solution comprising a biocompatible, synthetic, nucleophilic polymer component essentially free of human or bovine albumin and other biological molecules and including Poly-L-Lysine hydrobrornide having a number of active surface lysines of at least twenty (20) per 5000 M/W blended with a poly(ethylene glycol) (PEG) Amine having a functionality of four and a molecular weight of about 10,000 g/mole, wherein the weight-to-weight ratio of poly(ethylene glycol) (PEG) Amine to poly(ethylene glycol) (PEG) Succinimidyl Glutarate is selected to be about 0.7 to 1.0, and
instructions for use comprising
mixing the first and second solutions to form a synthetic hydrogel composition, and
applying the synthetic hydrogel composition by topically spraying the synthetic hydrogel composition onto a targeted wound site to promote wound healing.

11. A system according to claim 10
wherein the first solution is essential free of a buffer material.

12. A system according to claim 10
wherein the first solution comprising poly(ethylene glycol) (PEG) Succinimidyl Glutarate dissolved in Sterile Water for Injection USP (SW1) essentially free of a buffer material.

13. A system according to claim 10
wherein the second solution comprises the Poly-L-Lysine hydrobromide and poly(ethylene glycol) (PEG) Amine dissolved in HPLC-grade water for delivery that includes a buffer material.

14. A system according to claim 10
further including a dispensing unit that mixes the first and second solution and dispenses the mixture in situ through a dispensing tip, and
wherein the instructions for use direct use of the dispensing unit.

15. A system according to claim 14
wherein the dispensing unit is sized and configured as an integrated hand held device, or as an integrated hand held endoscopic device, or as an instrument system having mixing and dispensing units.

16. A system according to claim 14
wherein the dispensing tip is sized and configured as a needle, sprayer, or atomizer.

17. A system according to claim 10
further including one more auxiliary component comprising salicylate-based polyanhydride-esters formulated to degrade and release salicylic acid for anti-inflammatory effect; fillers, such as glucosarnine, glucosaminoglycans, and chondroitin sulfate; anti-inflamatory drugs; rapamycines and analogs, such as everolimus and biolimus; dexamethasone; M-prednisolone; interferon y-1b; leflunornide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG; plasticizers, including cellulose and/or non-reactive PEG compounds, such as PEG-hydroxyl compounds; therapeutic agents such as stem cells, antibodies, antimicrobials, collagens, genes, DNA, and other therapeutic agents; hemostatic agents such as thrombin, chitosan, diatomaceous earth (CELOX Material), silver, and/or GELFOAMO Material; growth factors; vasoconstrictors such as Ephinephrine (which includes hydroxyl groups (—OH) and an amine group (—NH) that could be incorporated to react with PEG-SG); lydocaine; and comparable compounds.

18. A method for treating burn tissue comprising
identifying a burn tissue site,
manipulating a dispensing unit to mix a first biocompatible, synthetic, electrophilic polymer solution with a second biocompatible, synthetic, nucleophilic polymer solution to form a synthetic hydrogel composition, the first solution being essentially free of human or bovine albumin and other biological molecules and comprising poly(ethylene glycol) (PEG) Succinirnidyl Glutarate having a functionality of four and a molecular weight of about 10,000 g/mole, the second solution also being essentially free of human or bovine albumin and other biological molecules and including Poly-L-Lysine hydrobrornide having a number of active surface lysines of at least twenty (20) per 5000 114/W, optionally blended with a poly(ethylene glycol) (PEG) Amine having a functionality of four and a molecular weight of about 10,000 g/mole, and
manipulating the dispensing unit to topically spray the synthetic hydrogel composition in situ onto the burn tissue site to provide at least one of the following treatments outcomes: use of the synthetic hydrogel composition as a hemostatic agent; and/or use of the synthetic hydrogel composition as a graft fixation agent; and/or use of the synthetic hydrogel composition to reduce need for postoperative wound care; and/or use of the synthetic hydrogel composition to reduce blood loss in an individual for whom blood transfusion is unacceptable.

19. A method comprising
providing a first solution comprising a biocompatible, synthetic, electrophilic polymer component including a multi-arm poly(ethylene glycol) (PEG) Succinimidyl Glutarate,
providing a second solution comprising a biocompatible, synthetic, nucleophilic polymer component essentially free of human or bovine albumin and other biological molecules and including a target amount of a polypeptide moiety having a number of active surface lysines of at least twenty (20) per 5000 M/W, the second solution, when mixed with the first solution, forming a synthetic hydrogel composition,
titrating the target amount and molecular weight of a polypeptide moiety in the synthetic hydrogel composition to change the physical properties of the synthetic hydrogel composition in terms of elasticity; and/or stability during storage prior to use (shelf life); and/or gelation time during use; and/or degradation time after use, and
instructing mixing of the first solution with the second solution in situ to form a synthetic hydrogel composition and applying the synthetic hydrogel composition onto a targeted tissue site to promote a therapeutic benefit due to the physical properties of the synthetic hydrogel composition.

20. A method according to claim 19
wherein the polypeptide moiety is Poly-L-Lysine hydrobromide.

21. A method comprising
providing a biocompatible, synthetic, electrophilic polymer component comprising a poly(ethylene glycol) (PEG) Succinimidyl Glutarate having a functionality of four and a molecular weight of about 10,000 g/mole, providing a biocompatible, synthetic, nucleophilic polymer component comprises a poly(ethylene glycol) (PEG) Amine having a functionality of four and a molecular weight of about 10,000 g/mole that upon mixing with the electrophilic polymer component undergoes a gelation process to form a hydrogel, and
delaying onset of the gelation process by blending with the nucleophilic polymer component a Poly-L-Lysine hydrobrornide having a molecular weight of greater than about 4000 g/mole.
